(19) United States
(12) Patent Application Publication
(10) Pub. No.: US 2019/0287681 A1
(43) Pub. Date: Sep. 19, 2019

(54) ARTIFICIAL INTELLIGENCE AND MACHINE LEARNING PLATFORM FOR IDENTIFYING GENETIC AND GENOMIC TESTS

(71) Applicant: GenomeSmart, Inc., Los Altos, CA (US)

(72) Inventors: Vandana Sanjay Sathe, Los Altos, CA (US); Sanjay Shrikrishna Sathe, Los Altos, CA (US)

(21) Appl. No.: 16/358,274

(22) Filed: Mar. 19, 2019

Related U.S. Application Data
(60) Provisional application No. 62/644,833, filed on Mar. 19, 2018.

(51) Int. Cl. G16H 50/20; G06N 20/20; G06N 5/04

(52) U.S. Cl. CPC G16H 50/20; G06N 5/046; G06N 20/20

(57) ABSTRACT

Improvements in genetic test identification are accomplished using a method and accompanying system that receives first input comprising recommendations for genetic tests given a plurality of different combinations of health-related variables and second input comprising information associated with available genetic tests. Based thereon, a set of rules comprising a plurality of mappings between the different combinations of health-related variables and the available genetic tests is generated. A classifier is trained using the set of rules as training data. Third input comprising a first combination of health-related variables is received, where the first combination of health-related variables is not included in the plurality of different combinations of health-related variables, provides the first combination of health-related variables as input to the classifier, and receives as output from the classifier, based on the input to the classifier, one or more recommended genetic tests from the available genetic tests.

### CROSS-REFERENCE TO RELATED APPLICATION

This application claims priority to and the benefit of U.S. Provisional Patent Application No. 62/644,833, filed on Mar. 19, 2018, the entirety of which is incorporated by reference herein.

### FIELD OF THE INVENTION

The present disclosure relates generally to artificial intelligence and machine learning technology, and, more specifically, to computer-implemented methods and accompanying systems for improving genetic and genomic test identification for individuals using intelligent health-related data processing and learning techniques.

### BACKGROUND

A genetic counselor is a health professional who typically is an advanced degree holder and is an expert in the understanding of genetic conditions and diseases. Today, millions of people cannot get access to a genetic counselor and thus cannot easily assess their risk of genetic disorders. In the current American health system, for example, unless a symptom is present, a physician will not refer a patient to a genetic counselor and, at times, the referral can come too late. Traditionally, patients expect doctors to identify any health risk they may have, instead of the patients doing it themselves. Increasingly, people are getting more health conscious and want to be proactive and participate in decision-making on their health. With the explosion in the direct to consumer (DTC) business in genetic testing, there is an increased interest in understanding one's genetic predisposition. There is also a significantly increased change of survival if a genetic disease like cancer is detected early. Early detection positively impacts health outcomes.

Each of us carries six to eight recessive gene mutations that when paired with a similar gene mutation in a partner, can cause a genetic disorder. Over 7,000 distinct rare diseases exist and approximately 80 percent are caused by faulty genes. The prevalence of all single gene diseases at birth is approximately 1/100. Cancer is a genetic disease that is caused by certain changes to genes. Additionally, “inherited genetic mutations” play a major role in about 5 to 10 percent of all cancers. An estimated one million people in the U.S., including men, carry one of the mutations of BRCA gene, and only about 10 percent are aware they do.

Genetic and genomic tests have applications in all areas of medicine, including cancer, chronic diseases and genetic disorders, and new tests are rapidly being introduced into clinical practice as science and technology advance. In the case of cancer, for example, genetic and genomic tests are used for screening, diagnosis, prognosis, and monitoring and treatment selection. Yet there is a paucity of genetic education and testing resources focused towards consumers. Today, there are thousands of genetic tests, each one targeted at addressing some specific genetic disorder. Selection of the tests is challenging. A typical search on the internet can lead you to incorrect and unreliable data. The available testing information is very complex and not focused towards patients, but more towards research and medical professionals.

Current sources of genetic and molecular testing are not comprehensive, and the content is not organized in a user-friendly manner for patients and clinicians. Many commercial clinical laboratories (ARUP, QUEST, MAYO CLINIC, GENEDx) and academic clinical laboratories in Stanford, Emory, and Baylor College of Medicine Medical Genetics Laboratories, and companies like Ambry Genetics, Genomic Health and Pharmgkb.org offer an extensive menu of molecular tests. Government websites like Genetic Testing Registry and professional organizations like AMP (Association Molecular Pathology) provide a test directory but do not provide information on newer tests and are not easy to navigate for people without a genetic background. Referring patients to genetic counselors is not solving the problem. Counselors cannot possibly handle what's coming. There are approximately 4,000 genetics counselors nationwide. Additionally, there are over 77,000 genetics test today with ten new tests being introduced into the market each week.

### BRIEF SUMMARY

In one aspect, a method for improving genetic test identification comprises receiving first input comprising recommendations for genetic tests given a plurality of different combinations of health-related variables; receiving second input comprising information associated with available genetic tests; generating a set of rules based on the first input and the second input, wherein the set of rules comprises a plurality of mappings between the different combinations of health-related variables and the available genetic tests; training a classifier using the set of rules as training data; receiving third input comprising a first combination of health-related variables, wherein the first combination of health-related variables is not included in the plurality of different combinations of health-related variables; providing the first combination of health-related variables as input to the classifier; and receiving as output from the classifier, based on the input to the classifier, one or more recommended genetic tests from the available genetic tests. Additional aspects include corresponding systems and non-transitory computer-readable media storing computer-executable instructions.

Various implementations of the foregoing can include one or more of the following features. A particular combination of health-related variables comprises age, ethnicity gender, personal medical history, and family medical history. The first input is received from a plurality of genetic counselors. The first input is structured into structured first input comprising generic paths that each lead to a recommendation of a specific genetic test, wherein generating the set of rules comprises providing the structured first input as input to a rule generation tool and receiving as output the set of rules. The second input is structured into structured second input comprising a plurality of correlations of gene/gene panels with different genetic conditions wherein generating the set of rules comprises providing the structured second input as input to a rule generation tool and receiving as output the set of rules. The genetic tests comprise genetic tests to identify hereditary cancer and/or tests associated with reproductive genetics.

In one implementation, fourth input comprising one or more sets of medical guidelines is received, and a plurality of scenarios is identified based on different combinations of health-related variables as applied to the one or more sets of medical guidelines, wherein generating the set of rules comprises generating a subset of rules for each scenario in the plurality of scenarios.

In another implementation, training the classifier using the set of rules comprises providing the set of rules as input to a decision tree classifier and applying a random forest algorithm.

In yet another implementation, a user interface configured to present a plurality of questions to a user to collect the first combination of health-related variables from a user is provided. The user interface can be configured to present the one or more recommended genetic tests to the user.

The details of one or more implementations of the subject matter described in the present specification are set forth in the accompanying drawings and the description below. Other features, aspects, and advantages of the subject matter will become apparent from the description, the drawings, and the claims.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the implementations. In the following description, various implementations are described with reference to the following drawings.

FIG. 1 depicts an example data flow into and out of one implementation of a rules engine for identifying relevant genetic tests.

FIG. 2 depicts example combinations of gender, ethnicity, and age.

FIG. 3 depicts a high-level architecture of a system for identifying relevant genetic tests, according to an implementation.

FIG. 4 depicts an example decision tree.

FIGS. 5-8 depict example user interface screens in one implementation of a genetic test identification platform.

FIGS. 9-11 depict example rules for identifying genetic tests to recommend.

### DETAILED DESCRIPTION

Described herein are methods and accompanying systems that implement a recommendations and matching engine to direct individuals to appropriate genetic tests based on the individuals' profiles. Using the personal medical history, family medical history, ethnicity and age of a user, the system generates a multitude of variables based on the possible combinations (e.g., 80 for age, 8 for ethnicity, and other variables for personal medical history and family history). The variables are provided as input to the system (in one implementation, into a machine learning algorithm), which provides as output the genetic and genomic tests which should be performed on the user. In generating this output, the system also considers national medical guidelines from bodies like NCCN (National Comprehensive Cancer Network), ACMG (American College of Medical Genetics), ACOG (American College of Obstetricians and Gynecologists), ASRM (American Society for Reproductive Medicine), and SMFM (Society for Maternal-Fetal Medicine). Tests identified for the user can be selected from a collection of available genomic and genetic tests, such as all available tests from the internet that are clinically approved for utility and validity.

To implement the above, the present disclosure describes a comprehensive platform which allows an individual to traverse the process of starting the test identification platform (e.g., via a website, mobile application, or other user interface), entering information over a brief assessment (e.g., a questionnaire that takes approximately seven minutes to complete), and, upon completing the assessment, instantly receiving a report with recommended tests. To address the difficulties in an individual's remembering and identifying information on family and personal medical history which may be relevant for genetic risk assessment, the platform includes modules which enable information flow between a physician and the individual for personal history, as well as a way to capture all unanswered questions on family history and send it back to the individual to check and upload. This is an important part of inputting correct information to order to get the right result.

The intelligence underlying the platform is enhanced by using training data associated with actual genetic counselors identifying what the recommended tests would be for a certain set of input by an individual. These data sets of input and recommended tests assist the machine learning algorithm in learning what is and is not important to consider in each such set of inputs. The resulting output of the platform is an assessment report for each individual which is substantially instantly produced after typically a seven minute assessment (set of questions). In some implementation, the report contains (1) suggested relevant types of tests; (2) simplifying information on how to read the results of the tests; (3) list of relevant labs including and pros and cons of tests; (4) estimated costs; (5) general insurance coverage criteria; (6) information about genetic testing itself, benefits and limitations of doing genetic testing; and (7) educational insights to help people understand the role and impact of genetics in one's family and life.

In one implementation, the platform is able to suggest genetic tests to identify common types of hereditary cancer, including brain, breast, colorectal, kidney/renal, stomach, thyroid, ovarian, pancreas, prostate, melanoma, and uterine. In another implementation, the platform can suggest genetic tests relating to reproductive genetics for couples who have natural conception or those using assisted reproductive technology and others with fertility issues, including carrier testing, fertility testing, recurrent pregnancy loss testing, pre-implantation genetic testing, pre-natal testing, and newborn testing. In a further implementation, the platform provides any one or more of pharmacogenetic testing insights, oncology care testing insights, heart health testing insights, rare disease testing insights, neurology/psychiatry testing insights, and microbiome.

### Section I: Genetic Test Matching Platform

In one implementation, the platform is an artificial intelligence/machine learning (AI/ML) based “patient to most appropriate genetic test matching platform,” which provides relevant, timely, concrete and actionable insights on which specific genetic tests need to be undertaken, based on specific guided inputs from health-conscious individuals or patients, enabling them to make informed decisions on prevention or treatment. Features and benefits of the platform include the following:

(a) AI platform which takes as input an individual's clinical and personal information and identifies relevant genetic tests, resulting in a “patient focused” customized, neutral source of relevant and reliable genetic testing information. The neutral aspect is particularly important, as with over 77,000 tests in the market today and with ten tests being introduced weekly, most companies are marketing their tests and medical establishments are partnering with one company or another to promote their tests. There is no central neutral resource thinking on behalf of the individual.

(b) Reduces complexity for patients: genetic tests themselves are very complex and providing a matching algorithm to match the thousands of genetic tests to the individuals personal profile and produce the right tests for each individual in seconds is a very complex task, let alone providing details on what those tests mean, insurance coverage on them, etc. This functionality is not something any individual can do with an internet-enabled computer or even a team of genetic counselors sitting together under one roof.

(c) Ongoing identification of relevant and reliable data sources of commercially available tests and constantly changing guidelines issued by professional organizations for use of these tests and additionally associated data regarding reimbursements from insurance companies.

(d) Ontology of continuously curated genetic test-related context and content is taken to build highly complex data sets.

(e) Additional supplemental ever-changing data on ancillary information associated with tests is also curated.

(f) “Self-learning” logic based on AI/ML to identify which genetic tests to present and which not to, using a logic/rules engine. AI/ML matching algorithms to match the individual profile (which includes their relevant personal medical history, family medical history, ethnicity and age) to the genetic tests database, based on understanding of the medical genetics and national medical guidelines and applications of genomic and genetic testing to the different medical specialties, including continuously aggregating data and interpreting information based on a certain set of questions as to what is the right test for the individual, and educating the patient to make an informed decision with assistance from a medical professional.

(g) Applicability to a variety of diseases, such as hereditary cancer and inherited disorders.

(h) Facilitation of patient education, which increases understanding of results and appropriate medical management.

(i) Deep and broad insights (e.g., explaining the subtle nuances and factors which need to be considered while choosing tests from the different options available) into all the tests available in the market in order for patients to make an informed decision.

(j) Simplified output including identified clinical test related information for patients.

(k) Scalability: the platform is able to scale to impact thousands of users in a very short period of time.

(l) Provides rich and relevant educational content in a simplified easy to understand way to increase awareness of genetic testing.

(m) Creates opportunities for on-demand genetic counseling services.

(n) Provides for building communities around genetic testing for each type of cancer and other diseases with deep research on each led by a top doctor/researcher.

(o) Proprietary data on individuals/patients and their inputted information, observed in aggregate over time, drives analytics and insights.

(p) Potential for global expansion.

Advantageously, the present disclosure provides for a comprehensive technique for curating relevant information and building a user-friendly platform of genetic and genomic tests from commercially available options and using a proprietary AWL based matching algorithm to narrow down a selection of tests, based on patient specifics, to those which are the most relevant as per the patient's or individual's clinical needs.

Based on the report produced, patients or other individuals who want to know if they are pre-disposed to certain conditions can have a more educated conversation with healthcare professionals (e.g., physicians, genetic counselors or oncologists) to better understand the interpretation of tests, insurance coverage, labs where testing is done and clinical utility of tests.

A more personalized and preventive approach to inherited conditions requires developing a broad genetic literacy for patients considering genetic testing. Understanding the availability, the clinical utility, and interpretation of genomic and genetic tests will help allow for more informed decisions and better outcomes for patients. Patients will be more empowered so that they can take a more proactive role in their healthcare and testing decisions, in essence catching things early and doing something about it.

The platform can utilize a database of clinically available tests from multiple scientific, clinical and commercial sources of data of both FDA cleared/approved and CLIA certified, as well as clinical biomarkers recommended by professional organizations guidelines like NCCN, ACMG & ACOG. Various guidelines that can be considered by the platform are listed the “Guidelines” section of this disclosure, below.

A genetic and genomic test database referenced by the platform can be curated and regularly updated to ensure relevance, reliability and currency, and can be checked by medical and genetic experts. Updates on an ongoing basis can include information from a range of credible and trusted sources including health agencies, government websites, corporate and scientific articles.

The AI/ML platform will help in understanding the different types of tests available and terms used for determining eligibility and utility so that appropriate test selection and interpretation can be determined. To facilitate this, besides guidance from professional organizations like NCCN and ACMG, a glossary of terms and also hyperlinks to the meaning of terms in simple language can also be included and made available through a device user interface.

As one example, currently, molecular tests are used in inherited cancer risk prediction for hereditary breast and ovarian cancer, colorectal cancer to assess risk in cancer patients as well as healthy individuals with relevant family history.

Although this disclosure uses cancer and reproductive genetics to demonstrate how the platform is used, it should be appreciated that the present solution can be used for a variety of diseases, including but not limited to (1) all types of cancer; (2) reproductive genetics (pre-natal testing, newborn screening, carrier testing); (3) predictive testing for cardiovascular, neurological disorders, and hereditary cancer; (4) infectious diseases; (5) inflammation (immune conditions); (6) rare diseases; and (7) pharmacogenomics.

The matching technology utilized by the AI/ML platform can condense thousands of available genetic tests from the internet into a short list of the most appropriate genetic test(s) for each individual in seconds. In one implementation, there are several components of the technology: comprehensive sourcing, semantic matching, and adaptive learning.

With comprehensive sourcing, thousands of genetic tests available in public domain and on paid sites on the internet are identified and considered by the platform in making testing recommendations.

Semantic matching accounts for context and intent, not just keywords. One component of the platform matching engine is ontology. An ontology defines the concepts, relationships, and other distinctions that are relevant for modeling a domain. In one implementation, the platform uses an ontology developed for the genetic testing domain, including (1) indication/purpose, i.e., the hierarchy and relationships between the concepts; (2) comprehensive information about the course of the disease, the recurrence risks and prognosis; and (3) an understanding of the clinical utility of tests and whether the tests suggested have sufficient scientific evidence based on clinical studies, research articles and subject matter experts. For example, this can involve identification of cancer susceptibility genes implicated in hereditary cancer, which are associated with inherited risk for cancers using scientific literature and national medical guidelines. In some implementations, the ontology is continuously curated by human genetic data experts in combination with the search and match technology and can consistently grow.

Another component of the platform matching engine is the query generation and concept extraction engine. In one implementation, a method of matching genetic test profiles with a patient profile, using the query generation and concept extraction engine, comprises the steps of (1) extracting from a patient profile a plurality of concepts corresponding to an ontology, e.g., personal medical history, family medical history, ethnicity and age, etc.; (2) generating a normalized patient profile (wherein the normalized patient profile includes the plurality of concepts as above); (3) forming a search query at least in part based on the normalized patient profile and the ontology; (4) submitting the search query to a source of genetic test databases; (5) receiving an initial batch of genetic test profiles potentially matching the patient profile from the source of genetic test profiles; (6) extracting from a genetic test profile among the initial batch of genetic test profiles at least a subset of the plurality of concepts corresponding to the ontology; (7) generating a normalized genetic test profile, wherein the normalized genetic test profile includes the at least a subset of the plurality of concepts; and (8) determining whether the normalized patient profile matches with the normalized genetic test profile.

The foregoing method creates a list of the most viable genetic tests based on patient profiles. Various criteria guide the choice of the most appropriate genetic tests for each patient, using an inherent logic/rules engine that evaluates the criteria. Examples of such criteria include:

To Identify Genetic Tests associated with Hereditary Cancer:
Personal medical history
   Type of cancer
   Age of diagnosis
      Sub-type (e.g., triple negative)
   Associated conditions
   Tumor testing results (e.g., colon, uterine)
Family medical history
   Type/s of cancer
   Patterns (e.g., which two cancers are together?)
   Age of cancer diagnosis (e.g., brain<18 years; gastric<40 years)
   Number of relatives with cancer history
   History of colon polyps (e.g., more or less than 10)
   History of family genetic mutations identified

To Identify Genetic Tests Associated with Reproductive Genetics
Personal medical history
   Age—maternal and paternal
   Ethnicity
   Pregnancy history
      Natural conception
      Assisted reproductive technology (e.g., IVF, sperm or egg donor)
      Fertility issues
      Recurring pregnancy loss
Family medical history
   Chromosomal disorders (e.g., Down syndrome)
   Birth defects
   Genetic disorders
      Blood disorders (e.g., thalassemia, sickle cell anemia)
      Cystic fibrosis, spinal muscular atrophy
   Blindness and deafness
   Heart defects

On the basis of the above patient/individual information collected, the platform can recommend whether the patient or other individual should receive genetic testing or not, and if so, then the platform can identify the appropriate test(s).

With respect to adaptive learning, the platform can constantly “self-learn” based on the initial intelligent ranking and machine-learning based rules engine, to understand and identify over time the right set of tests for each patient, based on patient and genetic testing profiles.

### Section II: Underlying Technology

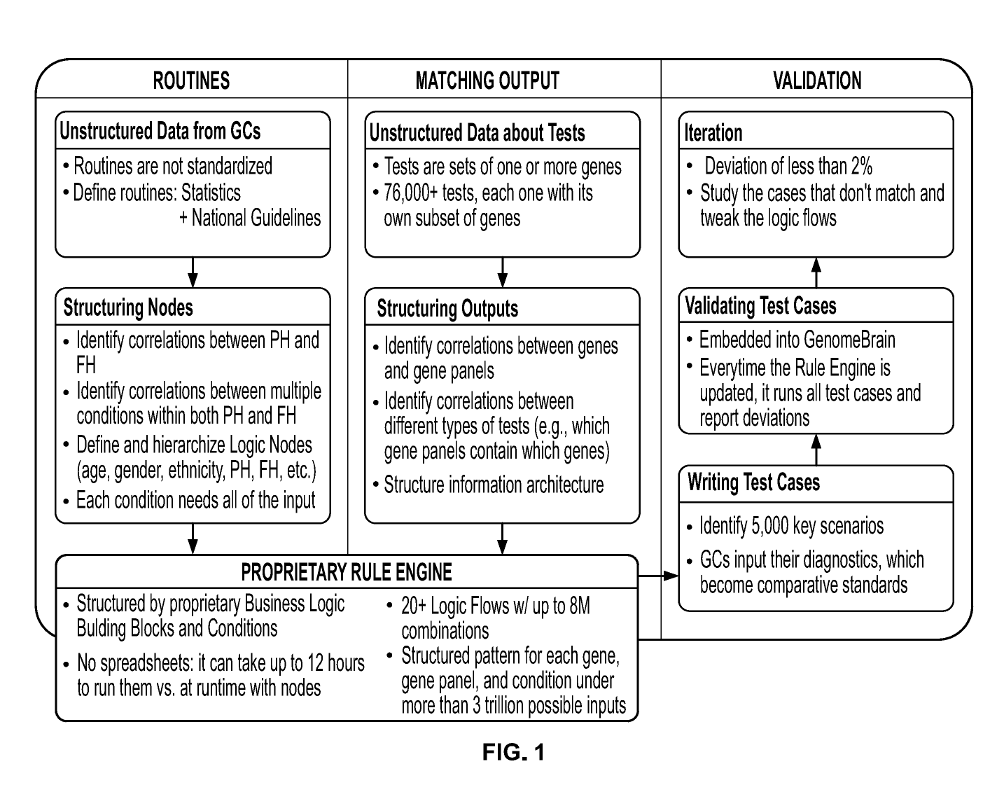

The platform uses machine learning to simulate the expertise of a genetic counselor and their daily routine in analyzing patients. Referring to FIG. 1, data from Routines and Matching Output is input into the ML/AI Proprietary Rule Engine to determine appropriate genetic tests for recommendation.

Unstructured Data from GCs (Genetic Counselors): Every genetic counselor has their own way to interpret the National Guidelines, including adding certain external factors like the counselor's experience and the geography they belong to and the type of patients they meet on daily basis. Based on all these parameters, the counselor analyzes what is to be recommended to a patient based on the patient's medical history. These routines are not standardized, and the data to use for the algorithm is unstructured. To train the platform, genetic counselors were asked for recommendations on the right tests for thousands of sets of input, ultimately resulting in the ML/AI engine predicting tests to be recommended for millions or trillions of combinations of health-related variables, including personal medical history (PH), family medical history (MH), ethnicity, age and gender. Input from the genetic counselors was further used to identify which questions that the counselors usually ask a patient to reach a certain conclusion and recommend a test.

Structuring Nodes: For any algorithm to work and develop, it is necessary to identify patterns and correlations between different parameters. The first step towards identifying such patterns and correlations is structuring the data in different nodes so that it can be further analyzed and converted in a way that it can lead towards a specific path. Structuring the aforementioned data from several genetic counselors is accomplished by removing the noise (i.e., the external parameters) and identifying a generic path and creating structured nodes that lead towards specific test recommendations, considering all relevant factors and guidelines.

Unstructured Data about Tests: Today, the available genetic tests are combinations of one or more genes. There are currently more than 76,000 tests available and the number is increasing on a daily basis. Many of these tests are interlinked and there are many providers performing these tests with different nomenclatures, leading to confusion. To address these issues, rather than identifying different providers and their tests, the platform instead identifies at a high-level all the gene and gene panel tests that can be recommended to the user.

Structuring Outputs: In this step, correlations between gene/gene panels and different genetic conditions are identified, and then the gene(s) and/or gene panel(s) that should be recommended in each scenario are determined. Once the correlations are identified, a structured information architecture stores this data so that the entire recommendation of genes and gene panels can be retrieved from this dataset in an efficient manner.

Still referring to FIG. 1, the Proprietary Rule Engine (ML/AI engine) ingests the data sets (user profile information, National Guidelines, and relevant available test data) and matches the user to the appropriate genetic tests. This exercise takes into account millions of combinations at run time to substantially instantly produce the result, i.e., a report with the recommendation of the “right” tests for the individual.

A multitude of variables are taken into account to produce the “right” results, i.e., recommended tests. In one implementation, in simple language, genetic recommendations are primarily based on the following basic features and parameters that can be provided by a user: gender (sex), age, ethnicity, personal health history (if any), and family health history (if any). Every addition or change in feature or parameter exponentially increases the number of possible combinations.

As noted above, unstructured data from genetic counselors can include sets of questions that the counselors ask to patients in order to arrive at the conclusion of which genetic tests to recommend. From those set of questions, patterns (e.g., flowcharts) are identified that each genetic counselor follows to move towards a test recommendation. When used in the platform, the questions are changed to have close-ended answers in order to manage combinations and arrive at a conclusion. The process is not necessarily an automatic process, as the inputs used to give the correct outputs are not constant. Thus, the variation in the user inputs, guidelines, and tests offered are constantly evolving so the algorithm will change with time to reflect those changes.

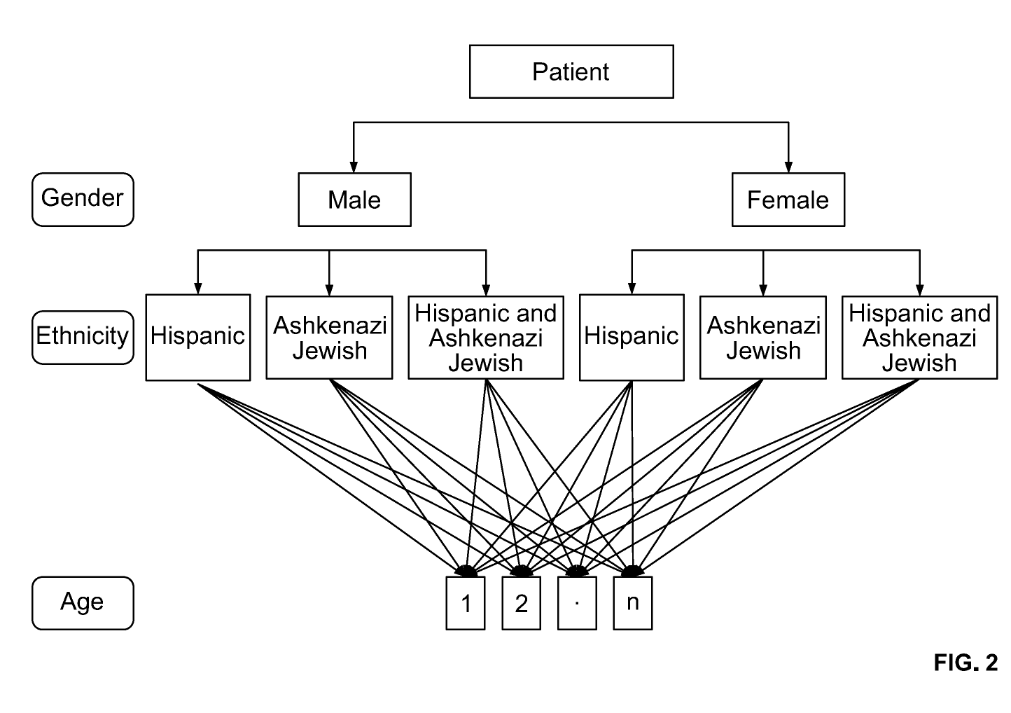

The number of variable combinations associated with identifying appropriate genetic tests can be considerable, resulting in significant computing cost. Representing the combinations as rows in a spreadsheet, for example, and processing the millions or trillions of rows to identify the applicable genetic test can take minutes of computing time, or more. In the example shown in FIG. 2, considering gender, 2 ethnicities, and age as the possible parameters, the number of combinations and complexities increases as moving down the tree. Complexity grows exponentially as additional parameters are added. To find the exact match(es) from trillions of combinations substantially instantly, while keeping computation cost to a minimum at runtime, the platform incorporates a proprietary framework, referred to as “GenomeBrain.”

GenomeBrain is a framework built as a combination of multiple open-source rule engines (e.g., JRules, Easy Rules) and internally built BLBBs (Business Logic Building Blocks) using available technologies (e.g., MongoDB, ElasticSearch), a JavaScript Object Notation (JSON) based parsing framework and a combination of machine learning algorithms, such as decision classifier and random forest algorithms.

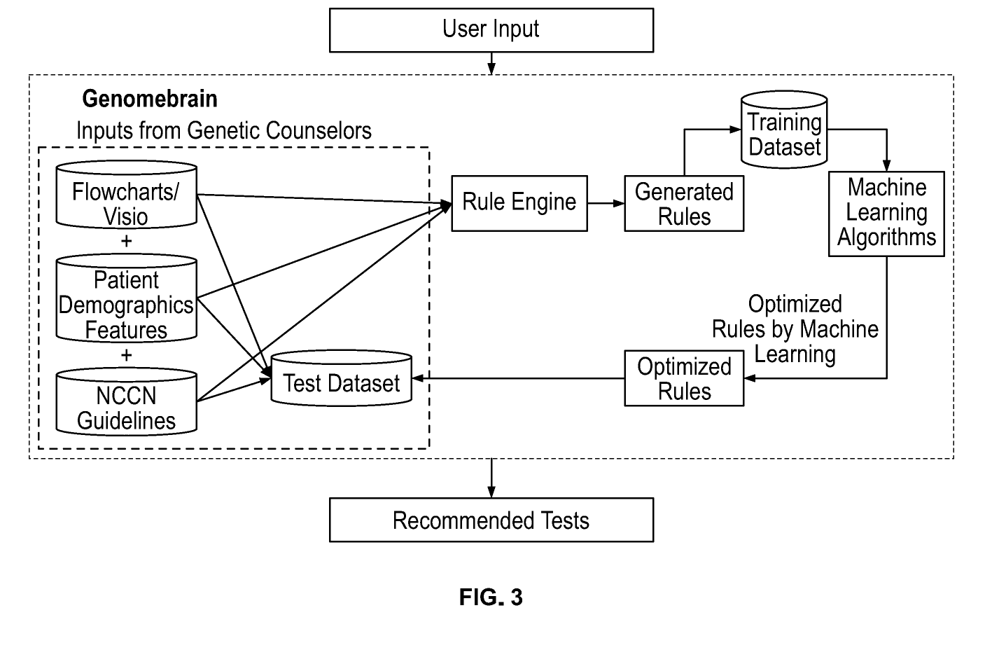

FIG. 3 depicts one implementation of GenomeBrain's architecture. The genetic counselor question patterns (e.g., flowcharts represented in MICROSOFT VISIO) are parsed using a JSON-based parser, and the resulting data is stored in a database (e.g., using MongoDB). Based on the combinations of personal and family history and patient demographic features, various scenarios are identified in light of medical guidelines (e.g. NCCN guidelines for cancer). The scenarios are then qualified into different buckets for further processing using rule creation tools (e.g., Easy Rules, JRules and BLBBs (proprietary JSON based framework)). One example set of rules is shown in Table 1. Row counts increase exponentially with the addition of every parameter. To find the exact match from these trillions of rows can be a tedious and an expensive task. Hence, it is necessary to optimize the already existing dataset and create an optimum path to the output.

TABLE 1

| Gender | Ethnicity | Age | Output |
|---|---|---|---|
| Male | Hispanic | 1 | Test 1, Test 2 |
| Male | Ashkenazi Jewish | 1 | Test 2, Test 3 |
| Male | Hispanic, Ashkenazi | 1 | Test 9, Test 11, Test |
| | Jewish | | 15 |
| Male | Hispanic | 2 | Test 2, Test 3 |
| Male | Ashkenazi Jewish | 2 | Test 9, Test 11, Test |
| | | | 15 |
| . . . | . . . | . . . | . . . |
| Female | . . . | . . . | . . . |

In one implementation, a Decision Tree Classifier supervised learning algorithm is used with available training data for solving regressions and classification problems. The rules created by the aforementioned tools are passed as training datasets for the machine learning ‘decision tree classifier’ algorithm. The output is then aggregated using a Random Forest algorithm which eventually optimizes the rules and recommends the tests.

Decision trees are prone to the problem of overfitting as the tree gets deep. To solve this problem, the Random Forest algorithm is used. A random forest is a collection of decision trees whose results are aggregated into one final result. Their ability to limit overfitting without substantially increasing error due to bias is why they are such powerful models.

For clarification, a “supervised learning algorithm” analyzes training data and produces an inferred function, which can be used for mapping new examples. An optimal scenario allows the algorithm to correctly determine the class labels for unseen instances. This requires the learning algorithm to generalize from the training data to unseen situations in a “reasonable” way. The main goal of a “regression” algorithm is the prediction of a discrete or a continuous value. “Classification” refers to predicting whether something falls into a target class. “Overfitting” is the phenomenon in which the learning system tightly fits the given training data so much that it would be inaccurate in predicting the outcomes of the untrained data.

Random forest is the prime example of ensemble machine learning method. In simple words, an ensemble method is a way to aggregate less predictive base models to produce a better predictive model. Random forests, as one could intuitively guess, assembles various decision trees to produce a more generalized model by reducing the notorious over-fitting tendency of decision trees.

Consider, for example, the above rule engine output table as the training dataset for the learning algorithm. In decision trees, for the process of predicting a class label for a record, the process starts from the root node of the tree. The values of the root attribute are compared with the record's attribute. On the basis of comparison, the branch corresponding to that value is followed and the process jumps to the next node. The process continues comparing the record's attribute values with other internal nodes of the tree until reaching a leaf node with a predicted class value. Thus, the modeled decision tree can be used to predict the target class or the value.

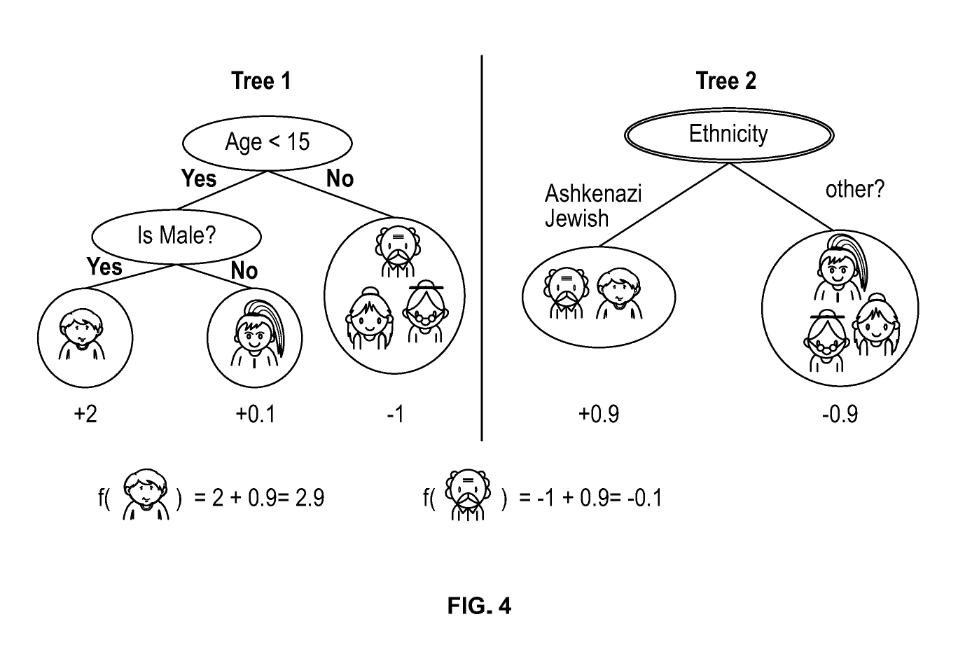

The decision tree model can be created as follows. Decision Trees follow Sum of Product (SOP) representation. FIG. 4 illustrates a prediction accounting for if patient's age plays a role in genetics? if patient's ethnicity plays a role in genetics? if patient's gender plays a role in genetics? from traversing for the root node to the leaf node. The SOP is also known as Disjunctive Normal Form. For a class, every branch from the root of the tree to a leaf node having the same class is a conjunction(product) of values, different branches ending in that class form a disjunction(sum).

The primary challenge in the decision tree implementation is to identify which attributes are necessary to consider as the root node and each level. Handling this is known as the attributes selection. Different attributes selection measures can be used to identify the attribute which can be considered as the root node at each level. Attribute selection measures can include information gain and Gini index.

If a dataset consists of “n” attributes, then deciding which attribute to place at the root or at different levels of the tree as internal nodes is a complicated step. Randomly selecting any node to be the root does not solve the issue and causes in low accuracy results. To address this, one can use a criterion like information gain, Gini index, etc. These criteria calculate values for every attribute. The values are sorted, and attributes are placed in the tree by following a particular order, e.g., the attribute with a high value (in case of information gain) is placed at the root. When using information gain as a criterion, attributes are assumed to be categorical, and when using Gini index, attributes are assumed to be continuous. Based on the Gini index or information gain calculations, a decision tree can be built. Attributes are placed on the tree according to their values.

Referring back to FIG. 1, validation of the results of the platform can be performed periodically. For example, with any update to a guideline, user questioning processes, or other logic, the platform re-executes all test cases with the new information and flags any exceptions. The platform thus learns to identify deviations and provide better results.

### Section III: Example Implementations

### EXAMPLE 1

### Use of Genetic Test Matching Platform: Hereditary Cancer

An example use of one implementation of the Al/ML platform will now be described with respect to identifying appropriate genetic tests relating to Hereditary cancer. To receive customized results for individuals and patients, the following data can be captured by the platform, e.g., by a potential test subject inputting the information into an electronic portal:

Example of Hereditary Cancer Assessment: The questions are dynamic (i.e., the following questions can change based on the answers to the prior questions). Additionally, the questions can be closed-ended with multiple choices.

Start by Asking Demographic Questions:

Age, Sex(biological), Ethnicity—Ashkenazi Jew, South Asian, Hispanic, Black/African American, South East Asian/Pacific Islander, White/Caucasian, Other. (For this example, the user selects Male, 64 years old, and Black and Hispanic ethnicity).

Do you have a Personal History of Cancer?—Yes or No.

Which Type(s) of Cancer(s) were you Diagnosed with? (on Selecting Yes, the following Choices are Presented):

Choose all that apply: Brain, Breast, Colorectal, Kidney/Renal, Melanoma, Pancreatic, Prostate, Skin (non-Melanoma), Stomach/Gastric, Thyroid, Uterine/Endometrial, Other (for each choice there is a sub choice where the specific age of diagnosis is asked).

On Choosing Prostate Diagnosed at the Age 35, the following is Presented:

Is your prostate cancer considered high grade or have a Gleason score of 7 or greater? (Here the platform can spell out the definition of complex terms, so in this case there is a tool tip for what is Gleason score).

There are three choices of answers—Yes or No or I don't know—need to check with a physician. (Often times people do not know or are not aware of details of the disease so it is important to ensure they input the right information and thus facilitate capture of all open questions. The answers can be provided to a hospital or physician portal so the physician can get back to the patient and the patient can confirm the information in order to get the assessment report).

On choosing No, move to the next question: Has/had cancer spread to Lymph nodes or other places in the body? (Again, the platform spells out the definition of complex terms, so in this case there is a tool tip for what is Lymph nodes).

On choosing Yes to Lymph nodes, the personal medical history input is completed and the platform moves to the family medical history section. (Getting family history is important as it can inform the platform of whether cancers in the family are caused by abnormal genes that have been passed from generation to generation. For purposes of the family history section, “family” can include blood relatives, e.g., parents, siblings, children, aunts, uncles, grandparents, nieces, nephews and first cousins on both sides of the family).

The First Question of Family History is: Do you have a Family History of Cancer?

On choosing Yes, the next question is: Which type(s) of cancer(s) has someone in your family been diagnosed with? Choose all that apply: Brain, Breast, Colon/Rectal, Kidney/Renal, Melanoma, Pancreatic, Prostate, Skin (non-Melanoma), Stomach/Gastric, Thyroid, Uterine/Endometrial, Other.

On choosing two cancers in the family, in this case “Breast” and “Colon/Rectal” cancers, the individual cancer questions will not be asked but instead the platform asks qualifying questions to check if the cancers could be hereditary. The next question in the family section presented is as follows: Do you have two close relatives on the same side of the family who have any of the following cancers? And at least one of them was diagnosed with the cancer at or before age 50? Breast, Ovarian, Pancreatic, Prostate, Melanoma, Colon/Rectal, Uterine, Stomach/Gastric, Kidney, Thyroid. There are four choices given here; Yes, No, Cannot find out, or I am not sure—need to check with family.

Do you have three close relatives on the same side of the family with either of following cancer diagnosed at any age? Breast, Ovarian, Pancreatic, Prostate, Melanoma, Colon/Rectal, Uterine, Stomach/Gastric, Kidney, Thyroid.

On choosing No to the previous question, the following question is presented: Do you have a close relative who was diagnosed with either of the following cancers? Ovarian, Pancreatic, Metastatic Prostate cancer (which has spread outside prostate gland), Breast cancer at or before age 45 years, Male breast cancer. There are four choices given here: Yes, No, Cannot find out, or I am not sure—need to check with family.

Then in the end ask two key questions are asked. The first is: Do you have a personal history of colon polyps? Options are Yes, No and Check with Physician. On choosing No, ask about the family history: Do you have a family history of colon polyps?

Then comes the next key question: Have you been found to have a cancer gene mutation? Again, there is a Yes or No choice.

On choosing No, go to the next question: Has any of your close family members been found to have a cancer gene mutation?

On choosing Yes, present a list of all the key cancer gene mutations to choose from: APC, ATM, EPCAM, BRCA1/2, MLH1, MSH2, CHEK2, MSH6, MUTYH, PTEN, NBN, TP53, PMS2, PALB2, BAPI, BRIP1, CDH1, CDK4, CDKN2A, FH, FLCN, MEN1, MET, RET, SDHA, SDHB, SDHC, SDHD, TSC1/2, VHL, OTHERS.

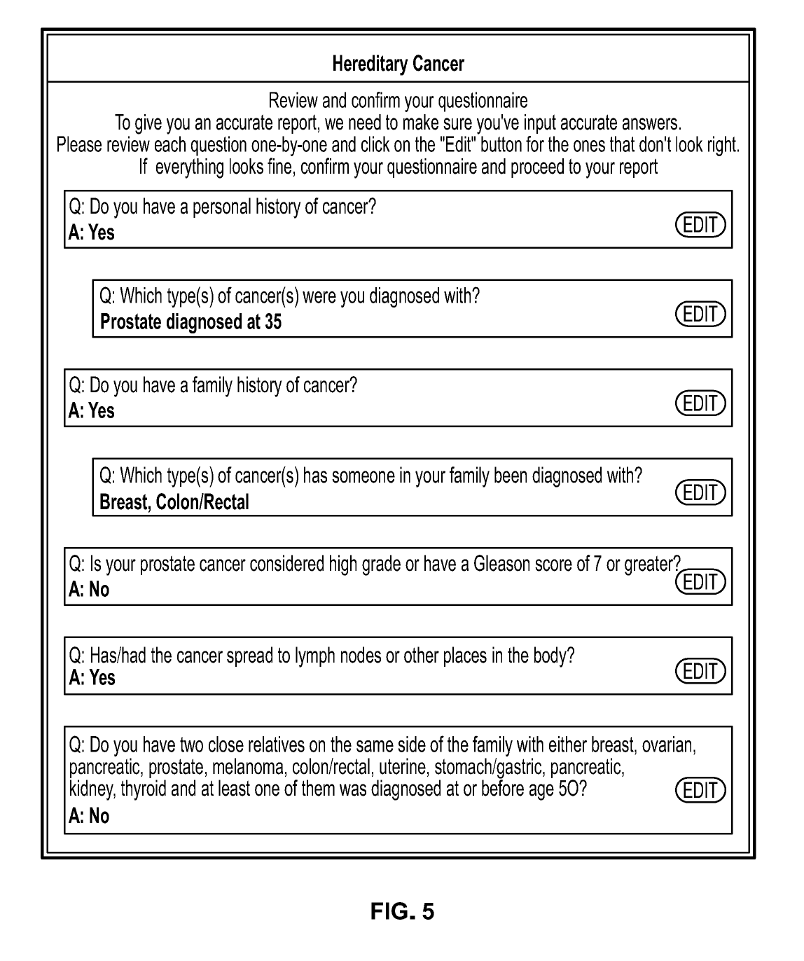

On choosing MLH1, the end of the assessment is reached. As depicted in FIG. 5, the user is given an opportunity to review all the answers to ensure an accurate assessment.

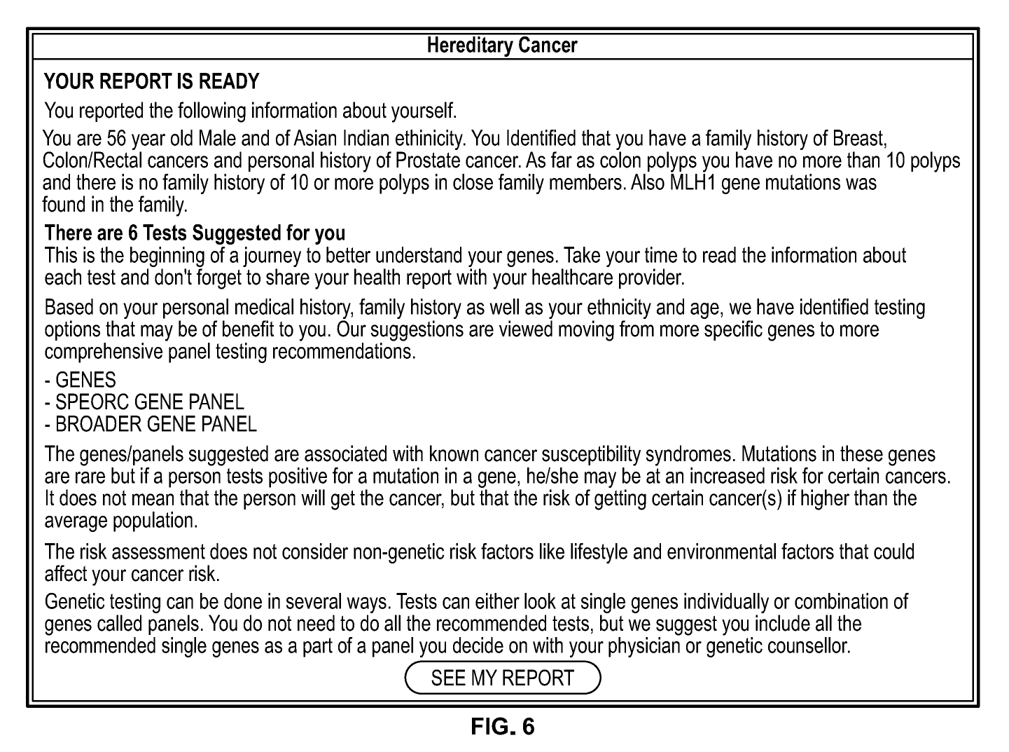

At the end of the summary of the answers given, the user is provided with a “see my report” button, and the first screen of the report is shown. An example of this is illustrated in FIG. 6. As shown, the user is provided with information that summarizes their inputs, informs them of the number of tests suggested for them (in this case, six), tell them how the platform will be presenting the suggested genes/gene panels, informs them that the risk assessment does not consider non-genetic risk factors like lifestyle and environmental factors that could affect cancer risk, and tells them how to take action on the report by explaining that they do not need to do all the recommended tests, but that they include all the recommended single genes as a part of a panel they decide on with their physician or genetic counselor.

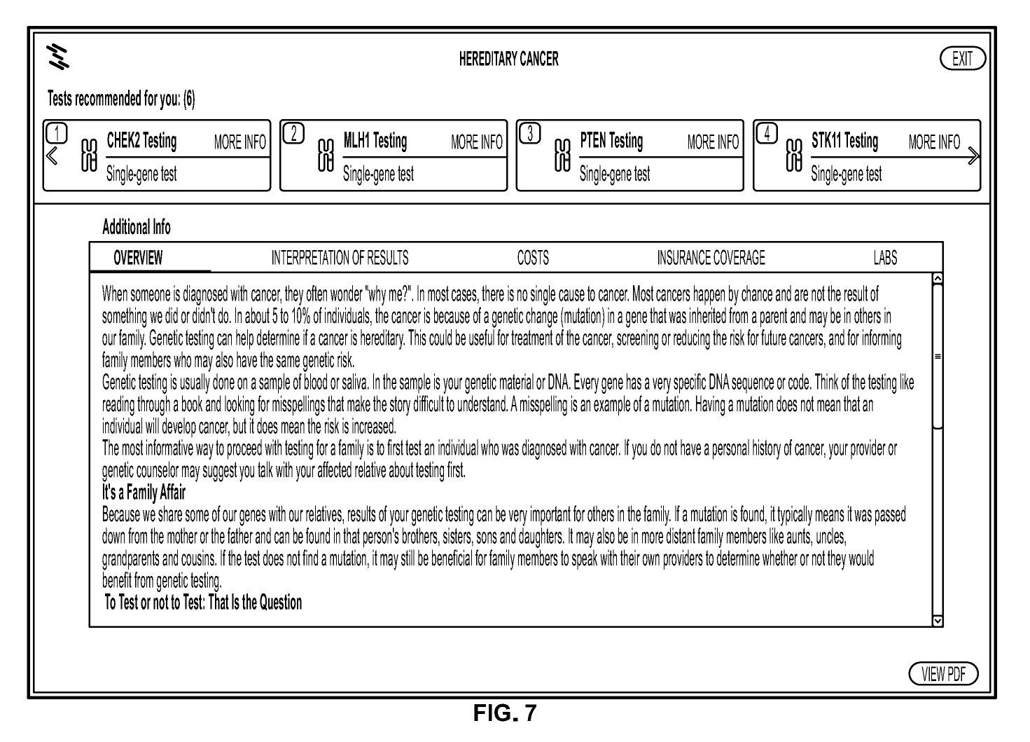

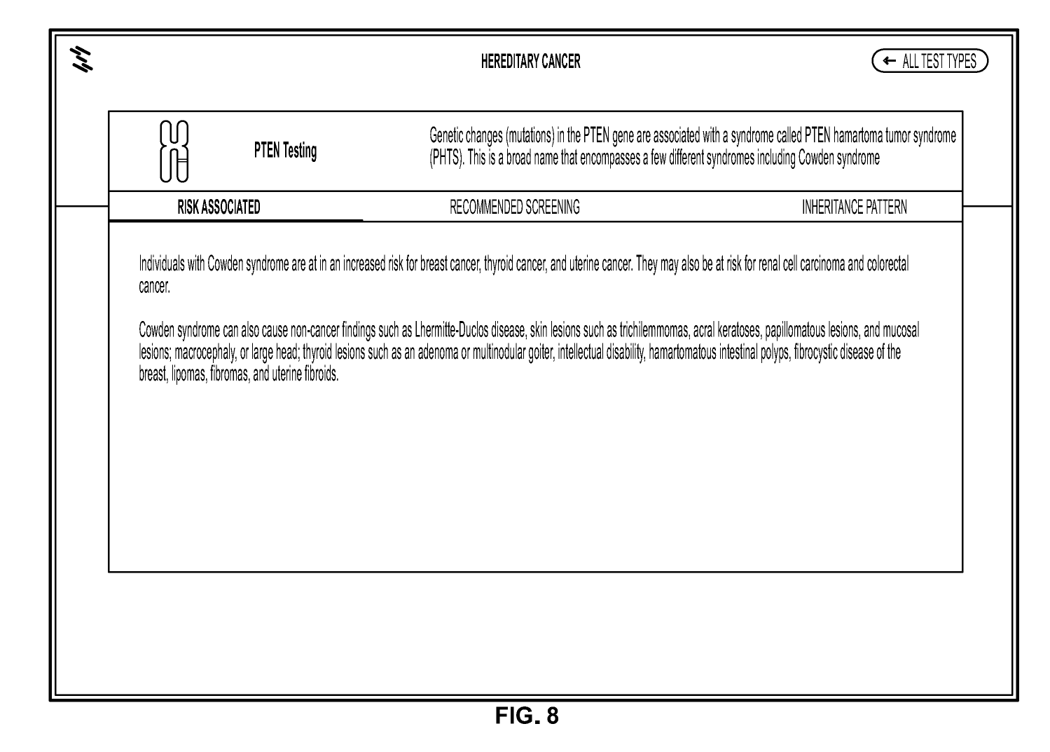

FIG. 7 depicts an example onscreen report that can be presented following the screen in FIG. 6. The onscreen report details the test recommendations on the top and provides general information below. If the user selects a particular gene, they can be shown more detailed information about the gene, as shown in FIG. 8. A report in a suitable format (e.g., PDF) can then be generated for the user containing the following: overview, details on how to interpret results when a genetic test is performed, associated cost, insurance coverage, labs, and the user's personalized test recommendations.

### Example 1: Backend Process

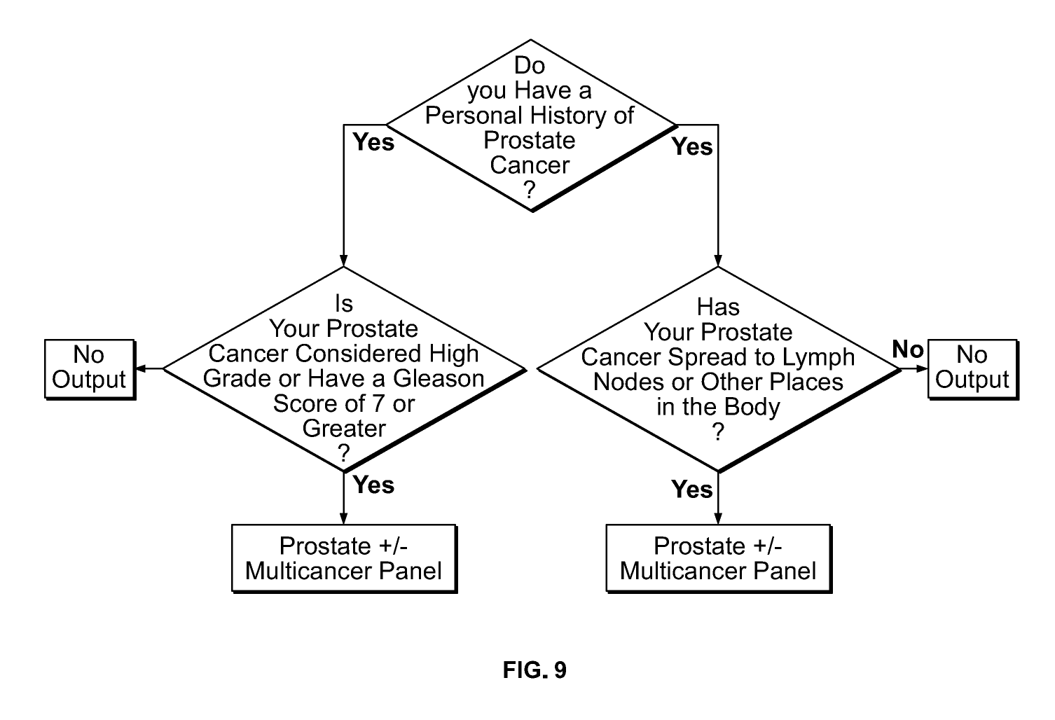

The details of the personal and family cancer history drive a set of actions on the back end of the platform. In this case, by the user's choosing prostate cancer in their personal history, the rules engine identifies the set of questions to be asked based on the answers and suggests the relevant genetic tests. FIG. 9 depicts a flowchart representing a procedural question-asking flow relating to prostate cancer that is followed by the rules engine.

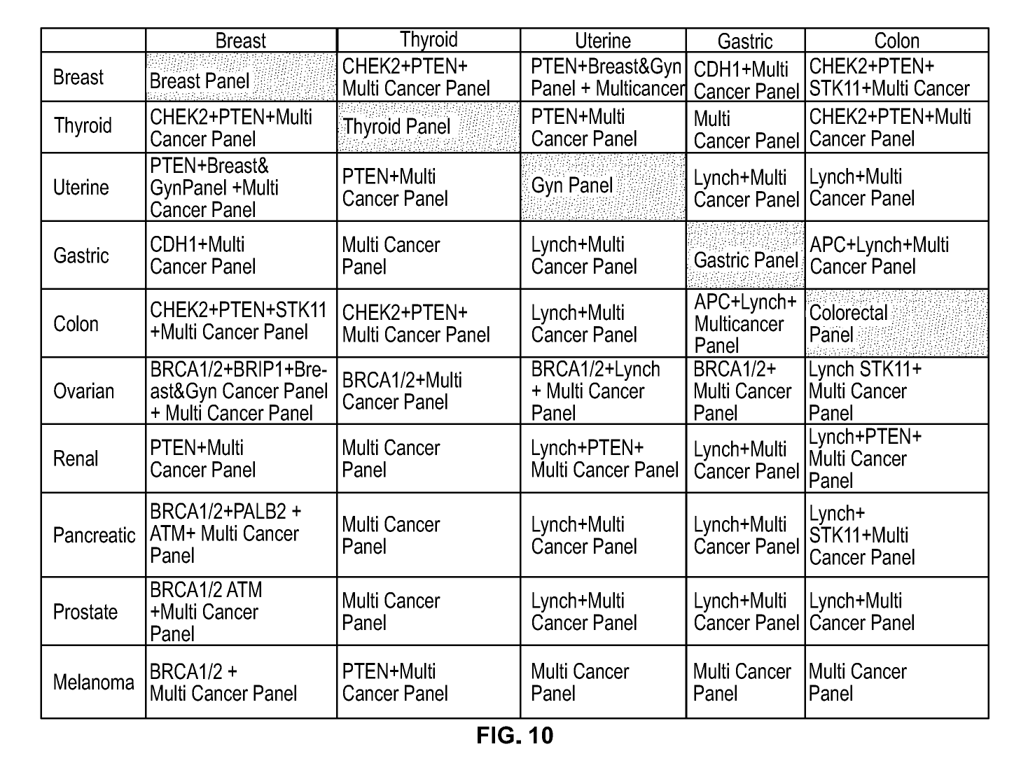

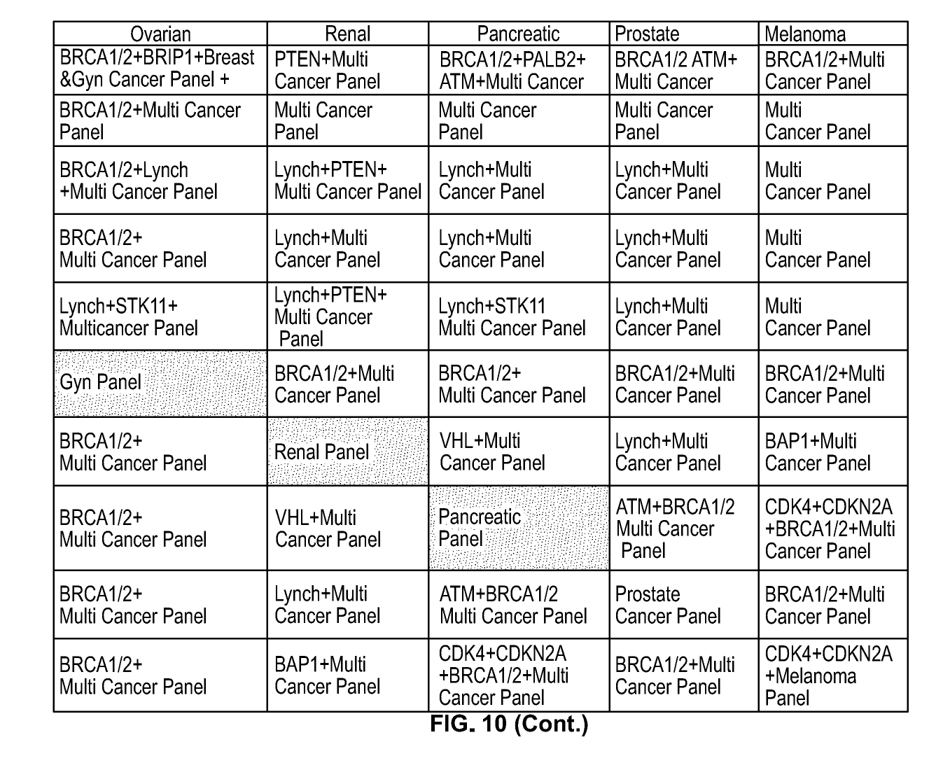

On the front end, the user moves through the assessment and reaches the family history section. If there is a history of only one cancer in the family, the rules engine is used to determine the next set of questions. In cases where there are two or more cancers in the family, a separate set of qualifying questions is asked to ascertain if the cancers truly are. On the back end, the system leverages a two-cancer combination rules engine, as shown in FIG. 10, which helps determine the right genetic testing recommendations. More specifically, genetic tests are selected based on the intersection of a row and column that correspond to the two cancers identified in the user's family history. If the user selects a family history of breast and colon/rectal cancer and answers yes to certain qualifying questions, the following genetic tests are suggested: CHEK2, PTEN, STK 11 and multi cancer panel.

In one implementation, two final questions presented to users of the platform are on polyps and gene mutations. If a user were to answer Yes to a family or personal history of polyps, testing suggestions would be based on rules pertaining to polyps. With respect to gene mutation, if the users answers affirmatively that there was a gene mutation in the family, then the specific gene that mutated is recommended to be tested. In the present example, the user chooses no personal or family history of polyps, but identifies the MLH1 mutation, so a test of the MLH1 gene is recommend in addition to any other tests.

### EXAMPLE 2

### Use of Genetic Test Matching Platform: Reproductive Genetics

An example use of one implementation of the AI/ML platform will now be described with respect to identifying appropriate genetic tests relating to reproductive genetics.

To receive customized results for individuals and patients, the following data can be captured by the platform, e.g., by a potential test subject inputting the information into an electronic portal:

Example of Reproductive Genetics Assessment: The questions are dynamic (e.g., the following questions can change based on the answers to prior questions). Additionally, the questions can be closed-ended with multiple choices.

Start by asking demographic questions: Age, Sex(biological), Ethnicity—Ashkenazi Jew, South Asian, Hispanic, Black/African American, South East Asian/Pacific Islander, White/Caucasian, Other. In this case, the user chooses Female, 37 years old, with Hispanic ethnicity.

The assessment is started with this question: Are you currently pregnant? With a Yes and No response. Based on the choice, the assessment takes the user through a different set of questions.

On selecting yes, the platform asks: What is your Estimated Due Date? The user provides a due date of Sep. 22, 2019.

Was this pregnancy achieved through in vitro fertilization (IVF)? Based on the choice the assessment takes the user through a different set of questions.

On selecting Yes to the previous question, the platform asks: Was there a sperm donor? If the answer is No, i.e., no sperm donor, then the following questions will not be asked to the user.

However, on selecting Yes to the sperm donor question, the platform asks: Was the sperm donor 40 years or older at the time of donation? Yes, No, Cannot find out, and I am not sure- need to check.

Upon selecting No, the assessment moves to the next question: What is the ethnicity of the sperm donor? The choices are: Ashkenazi Jew, South Asian, Hispanic, Black/African American, South East Asian/Pacific Islander, White/Caucasian, Other, cannot find out and I am not sure—need to check.

The next question is: Was there an egg donor. In this example, the user answers No, and no more egg donor questions are asked. The next question is: Did you use ICSI (Intracytoplasmic Sperm Injection)? (As with other questions which have complex terms, to help the user, there is a tool tip and, in this case, an explanation of what ICSI means).

On selecting Yes, the platform moves to the next question: Have you had two or more miscarriages?

On selecting No to this question, the next question is: Do you/your sperm donor have a family history of a recessive genetic condition? (Examples of some recessive genetic conditions include cystic fibrosis, sickle cell disease, spinal muscular atrophy, alpha thalassemia). (As with other questions which have complex terms, to help the user, the platform provides a tool tip explaining what recessive genetic condition means).

On saying No to the above question, the following question is asked: Do you have a history of unexplained ovarian insufficiency or failure?

On selecting No to the above question, the next question is: Does your sperm donor have a history of unexplained male infertility? Four choices are provided: Yes, No, Cannot find out, and I am not sure—need to check.

On saying No to this question, the next question is: Are you/your sperm donor a carrier of an X-linked condition? (Examples of an X-linked condition include Fragile X syndrome, Hemophilia, Duchenne Muscular Dystrophy, G6PD, X-linked ichthyosis).

On choosing No, the next question is: Do you/your sperm donor have/carry an autosomal dominant condition? Examples of autosomal dominant conditions include Huntington's disease, Marfan's disease, hereditary cancer (like Lynch syndrome, hereditary breast and ovarian syndrome).

On saying no to the previous question, the following question is asked: Do you/your sperm donor have a personal history, family history or prior pregnancy with a known genetic disorder?

On selecting no to the previous question, the following question is asked: Do you/your sperm donor or close relatives have any of the following conditions or pregnancy histories? (check all that apply): Chromosome abnormalities (such as Down syndrome); Neural tube defect (such as spina bifida or anencephaly); a blood disorder (hemophilia, thalassemia, sickle cell); Cystic fibrosis; a nerve or muscle disorder (neurofibromatosis, muscular dystrophy); a bone or skeletal disorder (achondroplasia or dwarfism); Heart defect at birth; Kidney abnormalities; Cleft lip/cleft palate; Intellectual disability; Blindness or deafness before age 18; Cannot find out; I am not sure; None.

In this example, the user selects Neural tube defect. This is the last question and now the platform shows the user a preview of all the answers they have given to make sure that the answers are correct. In addition, at the end of the summary of the answers given, the platform displays have a check box with the following message: “I have read all the answers and they are correct to the best of my knowledge.” On checking the box and clicking the “see my report” button, the first screen of the report is presented, in which the platform: specifies the number of tests recommended; provides details on which national medical organizations guidelines are used as a part of building the rules engine; and explains the actual recommended tests. It is also explained to the user that they do not need to do all the recommended carrier screening tests individually, but it is suggested they include all the genetic conditions as a part of a panel. One blood draw can test for all these genes together. It is further explained that prenatal genetic testing can provide information on whether the baby has certain genetic conditions. Both screening and diagnostic tests are provided, and the user is asked to select the ones right for them after discussing with their partner and physician.

The platform then present the on screen report, which highlights the tests which are relevant to the user in terms of testing. When the user clicks a particular test, onscreen details regarding the test are displayed.

### Example 2

### Backend Process

The details of the user's pregnancy history (natural conception vs. assisted reproductive technologies) and family history of genetic disorders drive a unique set of actions on the back end of the platform. There is a different flow for male users and female users. Further, the questions change based on whether the user/partner is pregnant or not. If the user is pregnant but has used an assisted reproductive technology, then the flow is further different from users who may be pregnant by natural conception.

In this case, by choosing that she is pregnant and used IVF, the rules engine identifies the set of questions to be asked and, based on the answers, suggests the relevant genetic tests.

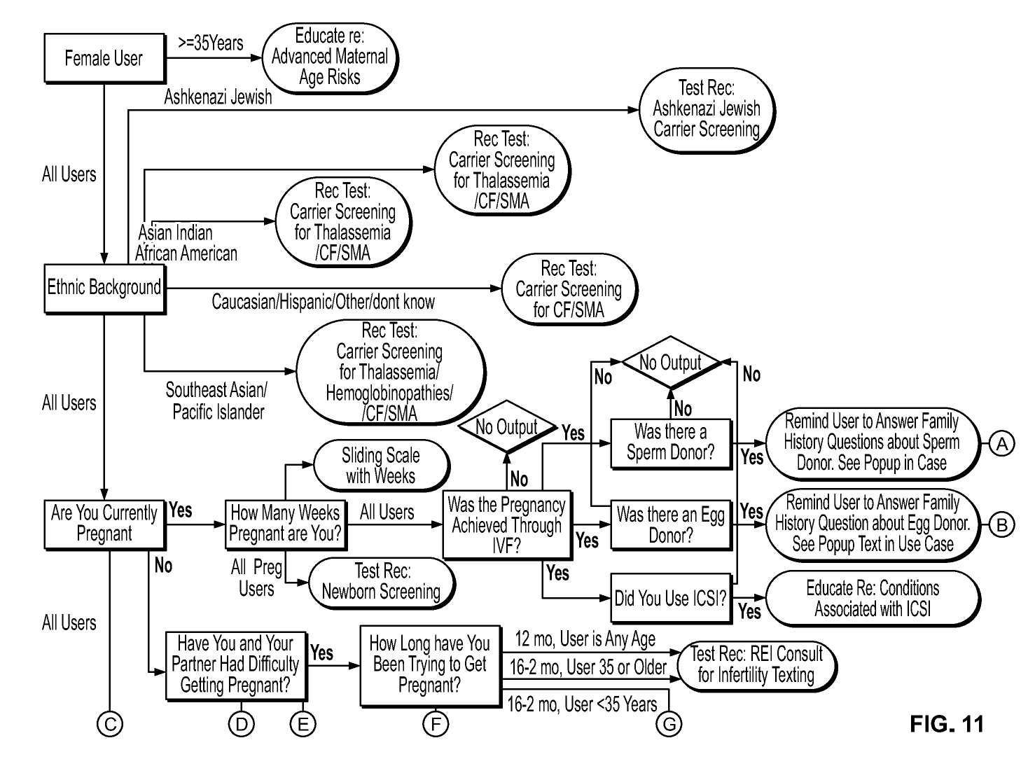

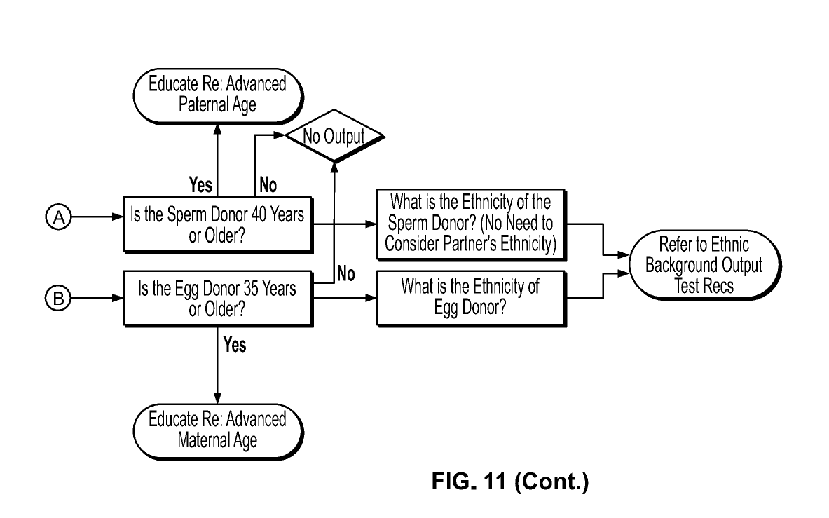

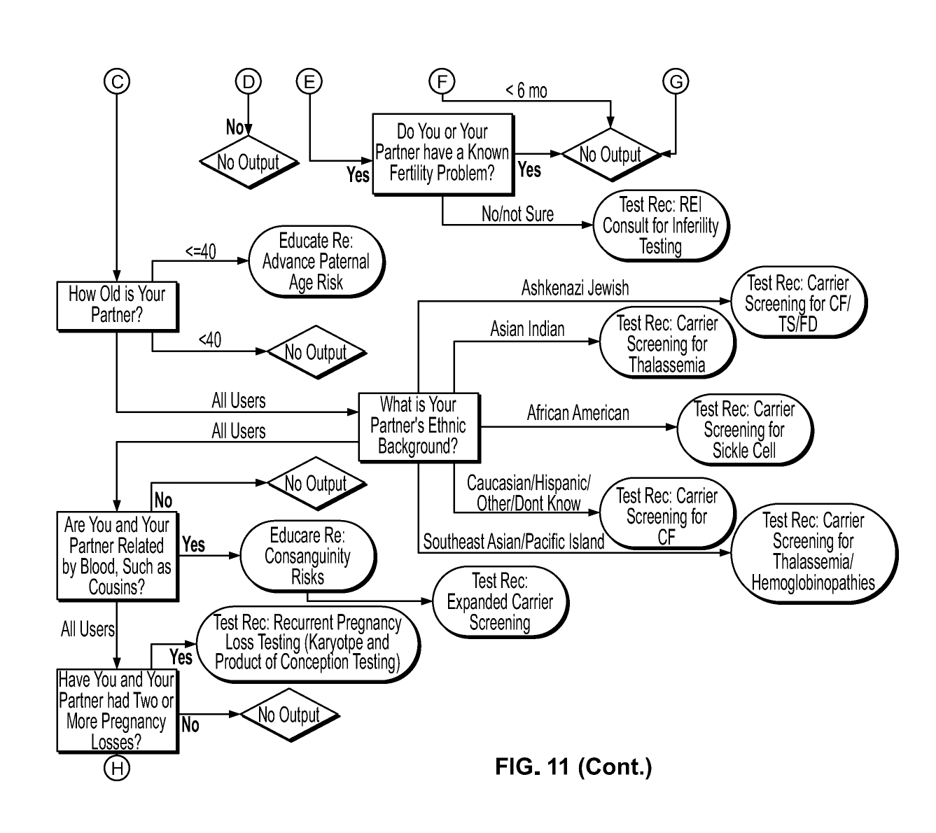

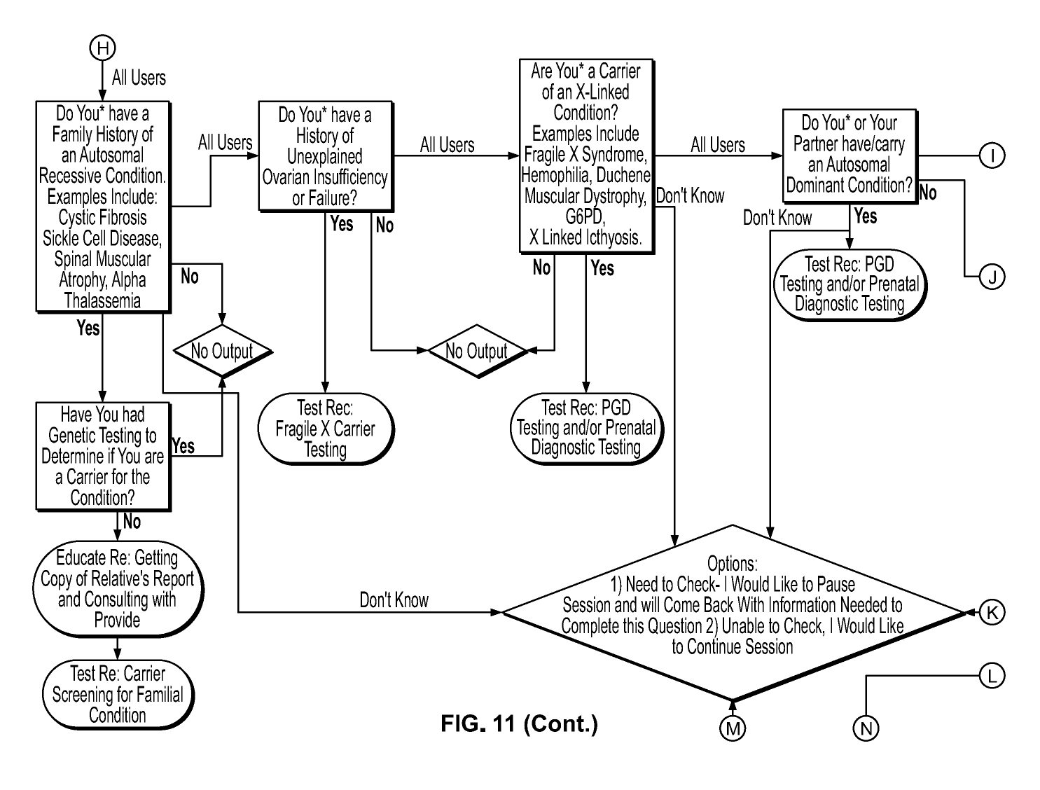

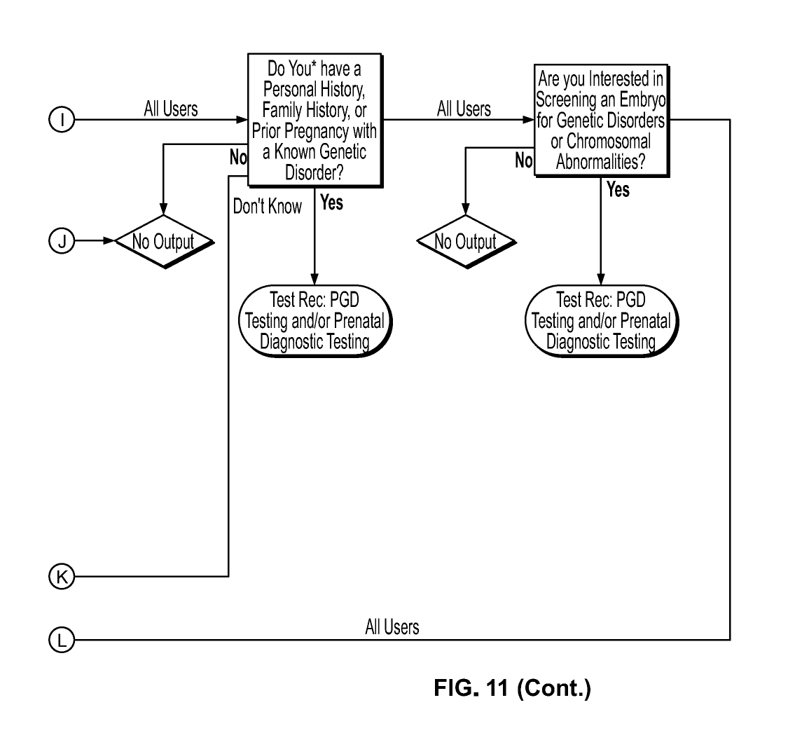

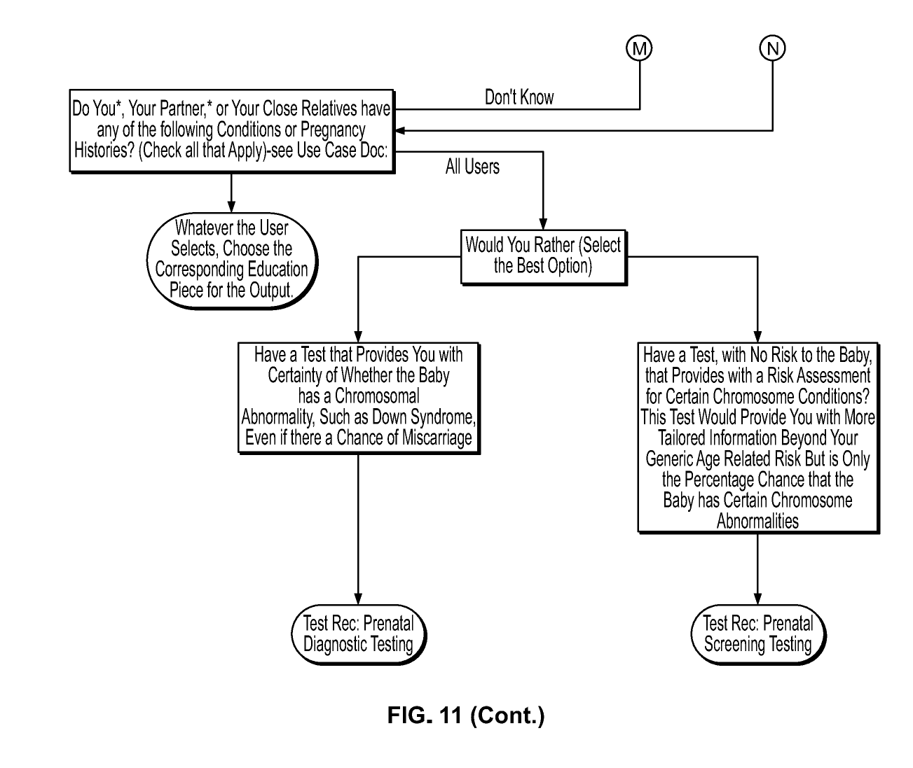

FIG. 11 depicts a flowchart of one implementation of a process flow used by the platform rules engine for questioning a female user regarding reproductive genetics. Based on the inputs provided by the user in this example and this process flow, the following tests are recommended to the user: Spinal Muscular Atrophy Carrier Screening, Thalassemia Carrier Screening, Cystic fibrosis Carrier Screening, State mandated newborn screening, Expanded newborn screening, Prenatal Screening Tests, Prenatal Diagnostic Tests (1st trimester Serum Screen, Anatomy scan (ultrasound), Quad Screen, Non-invasive prenatal screening, Chorionic Non-invasive prenatal screening, Chorionic Villus Sampling, Amniocentesis). The carrier tests were based on the ethnicity of the user and the prenatal tests were suggested based on the estimated due date. Further, as the user is over 35 years of age, which makes her pregnancy high risk, and the user reported neural defects as a family/past pregnancy history, a section is added to the report which provides the user with some education on these important topics.

### Example 3

### Consumer Education Platform

One of the key reasons people are not able to catch health issues early is the lack of awareness and education. In the case of cancer, it is important to become educated and exposed so that one can determine if one is at a higher risk. If so, one can change the general population age-based screening guidelines so that one can catch the cancer early or make changes to lifestyle to possibly even prevent it. In the case of reproductive genetics, carrier screening, pre-natal testing and in some cases pre-implantation genetic testing can possibly prevent or manage genetic disorders which may run in a family.

In one implementation, the platform includes an education platform focused on basic genetics, hereditary cancers, and reproductive genetics. The platform simplifies the understanding of this complex field, by providing information in a user-friendly way with simple language and graphics to illustrate concepts. The education platform can be constantly updated with new and relevant articles and is searchable so that users can access articles which may be of interest to them.

### Guidelines

Professional Society Guidelines—REPRODUCTIVE GENETICS

American College of Obstetricians and Gynecologists (ACOG). ACOG Practice Bulletin No. 78: hemoglobinopathies in pregnancy.

American College of Obstetricians and Gynecologists (ACOG). ACOG Practice Bulletin No. 138: inherited thrombophilias in pregnancy.

American College of Obstetricians and Gynecologists (ACOG). ACOG Practice Bulletin No. 200: early pregnancy loss.

American College of Obstetricians and Gynecologists (ACOG). ACOG Committee Opinion No. 640: cell free DNA screening for fetal aneuploidy.

American College of Obstetricians and Gynecologists (ACOG). ACOG Committee Opinion No. 690: carrier screening in the age of genomic medicine

American College of Obstetricians and Gynecologists (ACOG). ACOG Committee Opinion No. 691: carrier screening for genetic conditions.

American Society for Reproductive Medicine (ASRM). Evaluation and treatment of recurrent pregnancy loss: a committee opinion.

American Society for Reproductive Medicine (ASRM). Definitions of infertility and recurrent pregnancy loss: a committee opinion.

American Society for Reproductive Medicine (ASRM). Diagnostic evaluation of the infertile male: a committee opinion.

American College of Obstetricians and Gynecologists' Committee on Practice Bulletins—Obstetrics; Committee on Genetics; Society for Maternal-Fetal Medicine. Practice Bulletin No. 162: Prenatal Diagnostic Testing for Genetic Disorders.

American College of Obstetricians and Gynecologists' Committee on Practice Bulletins—Obstetrics, Committee on Genetics, and the Society for Maternal-Fetal Medicine. Practice Bulletin No. 163: Screening for Fetal Aneuploidy.

Professional Society Guidelines—HEREDITARY CANCER

Lynch Syndrome:

1. Ulmar A, et al. Revised Bethesda Guidelines for Hereditary Nonpolyposis Colorectal cancer (Lynch Syndrome) and Microsatellite Instability. J Natl Cancer Inst. 2004 February 18; 96 (4): 261-268.

2. Bethesda Guidelines

3. Amsterdam criteria

US Preventive Services Task Force Recommendations:

1. BRCA-Related Cancer: Risk Assessment, Genetic Counseling, and Genetic Testing. 2013 (currently being updated)

2. Prostate Cancer: Screening. May 2018

3. Breast Cancer Screening. 2016

4. Colorectal cancer screening. 2016

5. Ovarian Cancer Screening: 2018

6. Pancreatic Cancer Screening: 2004

Breast:

1. NCCN Genetic/Familial High-Risk Assessment: Breast and Ovarian. Version 3.2019.

2. NCCN Breast Cancer Risk Reduction. Version 1.2019.

3. NCCN Breast Cancer Screening and Diagnosis. Version 3.2018.

4. NSGC Practice Guideline: Risk Assessment and Genetic Counseling for Hereditary Breast and Ovarian Cancer. (Berliner, J. L., Fay, A. M., Cummings, S. A. et al. J Genet Counsel (2013) 22: 155.)

5. Oeffinger K C, Fontham E T H, Etzioni R, et al. Breast Cancer Screening for Women at Average Risk: 2015 Guideline Update From the American Cancer Society. JAMA. 2015 ;314(15):1599-1614 .

Ovarian

1. NCCN Genetic/Familial High-Risk Assessment: Breast and Ovarian. Version 3.2019.

2. NSGC Practice Guideline: Risk Assessment and Genetic Counseling for Hereditary Breast and Ovarian Cancer. (Berliner, J. L., Fay, A. M., Cummings, S. A. et al. J Genet Counsel (2013) 22: 155.)

3. Society of Gynecologic Oncology statement on risk assessment for inherited gynecologic cancer predispositions.

Colon:

1. NCCN Colorectal Cancer Screening. Version 1.2018.

2. NCCN Genetic/Familial High-Risk Assessment: Colorectal- Version 1.2018.

3. Wolf A, Fontham E, Church T, et al. Colorectal cancer screening for average-risk adults: 2018 guideline update from the American Cancer Society. CA: A Cancer Journal for Clinicians/Volume 68, Issue 4. 30 May 2018.

Pancreatic

1. NCCN Pancreatic Adenocarcinoma—Version 1.2019.

Prostate

1. NCCN Prostate Cancer—Version 4.2018.

2. Wolf A, Wender R, Etzioni R, et al. American Cancer Society Guideline for the Early Detection of Prostate Cancer: Update 2010. CA: A Cancer Journal for Clinicians/Volume 60, Issue 2.

Thyroid

1. NCCN Thyroid Carcinoma—Version 2.2018.

Uterine

1. NCCN Uterine Neoplasms—Version 2.2019.

Stomach/Gastric

1. NCCN Gastric Cancer—Version 2.2018.

Neuroendocrine and Adrenal Tumors

1. NCCN Neuroendocrine and Adrenal Tumors—Version 4.2018.

Melanoma

1. NCCN Uveal Melanoma—Version 1.2018.

2. NCCN Cutaneous Melanoma—Version 1.2019.

### Computer-Based Implementations

In some examples, some or all of the processing described above can be carried out on a personal computing device, on one or more centralized computing devices, or via cloud-based processing by one or more servers. In some examples, some types of processing occur on one device and other types of processing occur on another device. In some examples, some or all of the data described above can be stored on a personal computing device, in data storage hosted on one or more centralized computing devices, or via cloud-based storage. In some examples, some data are stored in one location and other data are stored in another location. In some examples, quantum computing can be used. In some examples, functional programming languages can be used. In some examples, electrical memory, such as flash-based memory, can be used.

An example computer system that may be used in implementing the technology described in this document includes a processor, a memory, a storage device, and an input/output device. Each of the components may be interconnected, for example, using a system bus. The processor is capable of processing instructions for execution within the system. In some implementations, the processor is a single-threaded processor. In some implementations, the processor is a multi-threaded processor. The processor is capable of processing instructions stored in the memory or on the storage device.

The memory stores information within the system. In some implementations, the memory is a non-transitory computer-readable medium. In some implementations, the memory is a volatile memory unit. In some implementations, the memory is a non-volatile memory unit.

The storage device is capable of providing mass storage for the system. In some implementations, the storage device is a non-transitory computer-readable medium. In various different implementations, the storage device may include, for example, a hard disk device, an optical disk device, a solid-date drive, a flash drive, or some other large capacity storage device. For example, the storage device may store long-term data (e.g., database data, file system data, etc.). The input/output device provides input/output operations for the system. In some implementations, the input/output device may include one or more of a network interface devices, e.g., an Ethernet card, a serial communication device, e.g., an RS-232 port, and/or a wireless interface device, e.g., an 802.11 card, a 3G wireless modem, or a 4G wireless modem. In some implementations, the input/output device may include driver devices configured to receive input data and send output data to other input/output devices, e.g., keyboard, printer and display devices. In some examples, mobile computing devices, mobile communication devices, and other devices may be used.

In some implementations, at least a portion of the approaches described above may be realized by instructions that upon execution cause one or more processing devices to carry out the processes and functions described above. Such instructions may include, for example, interpreted instructions such as script instructions, or executable code, or other instructions stored in a non-transitory computer readable medium. The storage device may be implemented in a distributed way over a network, such as a server farm or a set of widely distributed servers, or may be implemented in a single computing device.

Although an example processing system has been described, embodiments of the subject matter, functional operations and processes described in this specification can be implemented in other types of digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible nonvolatile program carrier for execution by, or to control the operation of, data processing apparatus. Alternatively or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. The computer storage medium can be a machine-readable storage device, a machine-readable storage substrate, a random or serial access memory device, or a combination of one or more of them.

The term “system” may encompass all kinds of apparatus, devices, and machines for processing data, including by way of example a programmable processor, a computer, or multiple processors or computers. A processing system may include special purpose logic circuitry, e.g., an FPGA (field programmable gate array) or an ASIC (application specific integrated circuit). A processing system may include, in addition to hardware, code that creates an execution environment for the computer program in question, e.g., code that constitutes processor firmware, a protocol stack, a database management system, an operating system, or a combination of one or more of them.

A computer program (which may also be referred to or described as a program, software, a software application, a module, a software module, a script, or code) can be written in any form of programming language, including compiled or interpreted languages, or declarative or procedural languages, and it can be deployed in any form, including as a standalone program or as a module, component, subroutine, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable computers executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Computers suitable for the execution of a computer program can include, by way of example, general or special purpose microprocessors or both, or any other kind of central processing unit. Generally, a central processing unit will receive instructions and data from a read-only memory or a random access memory or both. A computer generally includes a central processing unit for performing or executing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few.

Computer readable media suitable for storing computer program instructions and data include all forms of nonvolatile memory, media and memory devices, including by way of example semiconductor memory devices, e.g., EPROM, EEPROM, and flash memory devices; magnetic disks, e.g., internal hard disks or removable disks; magneto optical disks; and CD-ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's user device in response to requests received from the web browser.

Embodiments of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network. The relationship of client and server arises by virtue of computer programs running on the respective computers and having a client-server relationship to each other.

### Terminology

The phraseology and terminology used herein is for the purpose of description and should not be regarded as limiting.

The term “approximately”, the phrase “approximately equal to”, and other similar phrases, as used in the specification and the claims (e.g., “X has a value of approximately Y” or “X is approximately equal to Y”), should be understood to mean that one value (X) is within a predetermined range of another value (Y). The predetermined range may be plus or minus 20%, 10%, 5%, 3%, 1%, 0.1%, or less than 0.1%, unless otherwise indicated.

The indefinite articles “a” and “an,” as used in the specification and in the claims, unless clearly indicated to the contrary, should be understood to mean “at least one.” The phrase “and/or,” as used in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The use of “including,” “comprising,” “having,” “containing,” “involving,” and variations thereof, is meant to encompass the items listed thereafter and additional items.

Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

While this specification contains many specific implementation details, these should not be construed as limitations on the scope of what may be claimed, but rather as descriptions of features that may be specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous. Other steps or stages may be provided, or steps or stages may be eliminated, from the described processes. Accordingly, other implementations are within the scope of the following claims.

1. A computer-implemented method for improving genetic test identification, the method comprising:
receiving first input comprising recommendations for genetic tests given a plurality of different combinations of health-related variables;
receiving second input comprising information associated with available genetic tests;
generating a set of rules based on the first input and the second input, wherein the set of rules comprises a plurality of mappings between the different combinations of health-related variables and the available genetic tests;
training a classifier using the set of rules as training data;
receiving third input comprising a first combination of health-related variables, wherein the first combination of health-related variables is not included in the plurality of different combinations of health-related variables;
providing the first combination of health-related variables as input to the classifier; and
receiving as output from the classifier, based on the input to the classifier, one or more recommended genetic tests from the available genetic tests.

2. The method of claim 1, wherein a particular combination of health-related variables comprises age, ethnicity gender, personal medical history, and family medical history.

3. The method of claim 1, wherein the first input is received from a plurality of genetic counselors.

4. The method of claim 1, further comprising structuring the first input into structured first input comprising generic paths that each lead to a recommendation of a specific genetic test, wherein generating the set of rules comprises providing the structured first input as input to a rule generation tool and receiving as output the set of rules.

5. The method of claim 1, further comprising structuring the second input into structured second input comprising a plurality of correlations of gene/gene panels with different genetic conditions wherein generating the set of rules comprises providing the structured second input as input to a rule generation tool and receiving as output the set of rules.

6. The method of claim 1, further comprising:
receiving fourth input comprising one or more sets of medical guidelines; and
identifying a plurality of scenarios based on different combinations of health-related variables as applied to the one or more sets of medical guidelines,
wherein generating the set of rules comprises generating a subset of rules for each scenario in the plurality of scenarios.

7. The method of claim 1, wherein the genetic tests comprise genetic tests to identify hereditary cancer and/or tests associated with reproductive genetics.

8. The method of claim 1, wherein training the classifier using the set of rules comprises providing the set of rules as input to a decision tree classifier and applying a random forest algorithm.

9. The method of claim 1, further comprising providing a user interface configured to present a plurality of questions to a user to collect the first combination of health-related variables from a user.

10. The method of claim 9, wherein the user interface is further configured to present the one or more recommended genetic tests to the user.

11. A system for improving genetic test identification, the system comprising:
a processor; and
a memory storing computer-executable instructions that, when executed by the processor, program the processor to perform operations comprising:
receiving first input comprising recommendations for genetic tests given a plurality of different combinations of health-related variables;
receiving second input comprising information associated with available genetic tests;
generating a set of rules based on the first input and the second input, wherein the set of rules comprises a plurality of mappings between the different combinations of health-related variables and the available genetic tests;
training a classifier using the set of rules as training data;
receiving third input comprising a first combination of health-related variables, wherein the first combination of health-related variables is not included in the plurality of different combinations of health-related variables;
providing the first combination of health-related variables as input to the classifier; and
receiving as output from the classifier, based on the input to the classifier, one or more recommended genetic tests from the available genetic tests.

12. The system of claim 11, wherein a particular combination of health-related variables comprises age, ethnicity gender, personal medical history, and family medical history.

13. The system of claim 11, wherein the first input is received from a plurality of genetic counselors.

14. The system of claim 11, wherein the operations further comprise structuring the first input into structured first input comprising generic paths that each lead to a recommendation of a specific genetic test, wherein generating the set of rules comprises providing the structured first input as input to a rule generation tool and receiving as output the set of rules.

15. The system of claim 11, wherein the operations further comprise structuring the second input into structured second input comprising a plurality of correlations of gene/gene panels with different genetic conditions wherein generating the set of rules comprises providing the structured second input as input to a rule generation tool and receiving as output the set of rules.

16. The system of claim 11, wherein the operations further comprise:
receiving fourth input comprising one or more sets of medical guidelines; and
identifying a plurality of scenarios based on different combinations of health-related variables as applied to the one or more sets of medical guidelines,
wherein generating the set of rules comprises generating a subset of rules for each scenario in the plurality of scenarios.

17. The system of claim 11, wherein the genetic tests comprise genetic tests to identify hereditary cancer and/or tests associated with reproductive genetics.

18. The system of claim 11, wherein training the classifier using the set of rules comprises providing the set of rules as input to a decision tree classifier and applying a random forest algorithm.

19. The system of claim 11, wherein the operations further comprise providing a user interface configured to present a plurality of questions to a user to collect the first combination of health-related variables from a user.

20. The system of claim 19, wherein the user interface is further configured to present the one or more recommended genetic tests to the user.
